(19) United States
(12) Patent Application Publication
(10) Pub. No.: US 2006/0194858 A1
(43) Pub. Date: Aug. 31, 2006

(54) Preparation of crude candesartan cilexetil

(76) Inventors: Omer Malachi, Rehovot (IL); Ziv Kurgan, Beer-Sheva (IL)

Correspondence Address: KENYON & KENYON LLP, ONE BROADWAY, NEW YORK, NY, 10004, US

(21) Appl. No.: 11/333,875

(22) Filed: Jan. 17, 2006

Related U.S. Application Data
(60) Provisional application No. 60/643,937, filed on Jan. 14, 2005.

(51) Int. Cl. A61K 31/4184; C07D 403/02

(52) U.S. Cl. 514/381; 548/253

(57) ABSTRACT

A process for preparing candesartan cilexetil comprising heating a solution of trityl candesartan cilexetil in a water immiscible solvent in the presence of at least one C₁-C₄ alcohol and a first portion of water; combining the solution with a second portion of water to obtain a two-phase system; and recovering candesartan cilexetil.

This application claims the benefit of U.S. Provisional Application No. 60/643,937, filed on Jan. 14, 2005, hereby incorporated by reference.

### FIELD OF INVENTION

The invention encompasses processes for preparing crude candesartan cilexetil.

### BACKGROUND OF THE INVENTION

Candesartan is a potent, long-acting, selective AT₁, subtype angiotensin II receptor antagonist. Candesartan is a useful therapeutic agent for treating circulatory system diseases such as hypertensive diseases, heart diseases (e.g. hypercardia, heart failure, cardiac infarction, etc.), strokes, cerebral apoplexy, and nephritis, among others. Candesartan meets the requirement of high potency but it is poorly absorbed when administered orally. Therefore, the prodrug candesartan cilexetil was developed. During absorption from the gastrointestinal tract candesartan cilexetil is rapidly and completely hydrolyzed to candesartan.

The chemical name for candesartan is: 2-ethoxy-1-[[2′-(1H-tetrazol-5-yl)biphenyl-4-yl]methyl]-1H-benzimidazole-7-carboxylic acid, whereas candesartan cilexetil is (±)-1-[[(cyclohexyloxy)carbonyl]oxy]ethyl-2-ethoxy-1-[[2′-(1H-tetrazol-5-yl)[1,1′biphenyl]-4-yl]methyl]-1H-benzimidazole-7-carboxylate. Candesartan cilexetil is a white to off-white powder and is sparingly soluble in water and in methanol. Although candesartan cilexetil contains an asymmetric center in the ester portion of the molecule, it is sold as the racemic mixture.

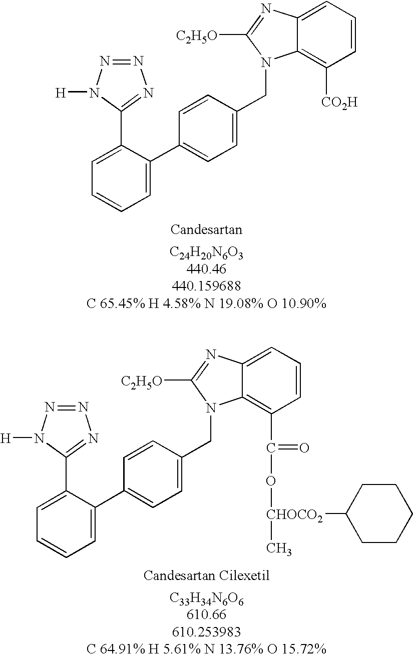

Candesartan plays an important role in blocking vasoconstriction by inhibiting a peptide, Angiotensin II. This peptide is formed from angiotensin I in a reaction catalyzed by angiotensin-converting enzyme (ACE, kininase II). Angiotensin II aids in maintaining constant blood pressure despite fluctuations in a person's state of hydration, sodium intake and other physiological variables, as well as performing the regulatory task of inhibiting excretion of sodium by the kidneys, inhibiting norephedrin reuptake and stimulating aldosterone biosynthesis. It is the principal pressor agent of the renin-angiotensin system, causing vasoconstriction, stimulation of synthesis and release of aldosterone, cardiac stimulation, and renal reabsorption of sodium. Candesartan blocks the vasoconstrictor and aldosterone secreting effects of angiotensin II by selectively blocking the binding of angiotensin II to the AT₁ receptor in many tissues, such as vascular smooth muscle and the adrenal gland. By inhibiting angiotensin II binding to AT₁, receptors, candesartan disrupts the vasoconstriction mediated by AT₁ receptors. Blocking vasoconstriction by angiotensin II has been found to be beneficial to patients with hypertension. The U.S. Food and Drug Administration has approved candesartan for the treatment of hypertension alone or in combination with other antihypertensive agents.

A method of preparing candesartan cilexetil is disclosed in U.S. Pat. No. 5,196,444. There, candesartan cilexetil is produced by the reaction of trityl candesartan with cyclohexyl 1-iodoethyl carbonate and hydrochloric acid. The candesartan cilexetil is recovered from the reaction mixture by extraction with ethyl acetate and water.

U.S. Pat. No. 5,578,733 (“'733 patent”) discloses a method of preparing candesartan cilexetil under substantially anhydrous conditions. The '733 patent discloses that preparing candesartan cilexetil under substantially anhydrous conditions is preferable to aqueous conditions because under anhydrous conditions, “the decomposition reaction is remarkably inhibited even when the starting N-protected tetrazolyl compound has a partial structure liable to undergo hydrolysis under acidic conditions, thus insuring a high reaction yield of the objective tetrazolyl compound.” '733 patent, col. 12, 11. 33-39.

The therapeutic effectiveness of candesartan cilexetil has created a need for additional efficient synthetic routes to the product. To address this need, the invention provides a process for preparing candesartan cilexetil.

### SUMMARY OF THE INVENTION

In one embodiment, the invention encompasses a process for preparing candesartan cilexetil comprising: heating a solution of trityl candesartan cilexetil in a water immiscible solvent in the presence of at least one C₁-C₄ alcohol and a first portion of water; combining the solution with a second portion of water to obtain a two-phase system; and recovering candesartan cilexetil.

Preferably, the C₁-C₄ alcohol is methanol, ethanol, propanol, isopropanol, butanol, or 2-butanol. More preferably the C₁-C₄ alcohol is methanol. Preferably, the C₁-C₄ alcohol is present in an amount of about 4 ml/g to about 12 ml/g of the trityl candesartan cilexetil. More preferably, the C₁-C₄ alcohol is present in an amount of about 6 ml/g of the trityl candesartan cilexetil.

Preferably, the water immiscible solvent is at least one of C₁₋₄ halo-hydrocarbons, C₆₋₁₀ aromatic hydrocarbons, linear or cyclic C₂₋₅ alkyl ethers, C₁₋₆ esters, C₃₋₅ ketones, C ₁₋₅ amides, or carbonates. More preferably, the water immiscible solvent is methylene chloride, ethyl acetate, or toluene. Most preferably, the water immiscible solvent is toluene. Preferably, the water immiscible solvent is present in an amount of about 1 ml/g to about 6 ml/g of the trityl candesartan cilexetil. More preferably, the water immiscible solvent is in an amount of about 3 ml/g of the trityl candesartan cilexetil.

Preferably, the first portion of water is present in an amount of at least about 0.5 mole per mole of trityl candesartan cilexetil. More preferably, the first portion of water is present in an amount of about 2 mole equivalents of the trityl candesartan cilexetil.

Preferably, the second portion of water is present in an amount of about 0.5 ml/g to about 5 mL/g of the trityl candesartan cilexetil. More preferably, the second portion of water is added in an amount of about 4 ml/g of the trityl candesartan cilexetil.

### DETAILED DESCRIPTION OF THE INVENTION

The invention encompasses processes for preparing candesartan cilexetil. The processes of the invention advantageously avoid distillation of the solvent. Distillation causes decomposition of candesartan cilexetil, which is temperature-sensitive, and, therefore, may reduce the yield of candesartan cilexetil. Thus, distillation is undesirable on industrial scale production.

In one embodiment, the process for preparing candesartan cilexetil comprises: heating a solution of trityl candesartan cilexetil in a water immiscible solvent in the presence of at least one C₁-C₄ alcohol and a first portion of water; combining the solution with a second portion of water to obtain a two-phase system; and recovering candesartan cilexetil.

The water immiscible solvent is capable of dissolving the trityl candesartan cilexetil. Suitable water immiscible solvents include, but are not limited to, at least one of C₁₋₄ halo-hydrocarbons, C₆₋₁₀ aromatic hydrocarbons, linear or cyclic C₂₋₅ alkyl ethers, C₂₋₆ esters, C₃₋₅ ketones, C₁₋₅ amides, or carbonates. Preferred solvents include methylene chloride, ethyl acetate, or toluene, and most preferably, the water immiscible solvent is toluene. Preferably, the water immiscible solvent is present in an amount of about 1 ml/g to about 6 ml/g of trityl candesartan cilexetil, and more preferably about 3 ml/g.

Any alcohol capable of deprotecting trityl candesartan cilexetil may be used. Suitable C₁-C₄ alcohols include, but are not limited to, at least one of methanol, ethanol, propanol, isopropanol, butanol, or 2-butanol. The preferred alcohol is methanol. The alcohol may be in any amount sufficient to promote the reaction. Preferably, the alcohol is in an amount of about 4 ml/g to about 12 ml/g of trityl candesartan cilexetil, and more preferably about 6 ml/g.

The first portion of water is added in an amount of at least about 0.5 mole per mole of the trityl candesartan cilexetil, preferably about 2 mole per mole of the trityl candesartan cilexetil.

The solution may be heated at any temperature and for any amount of time sufficient to deprotect the trityl candesartan cilexetil and form candesartan cilexetil. Preferably, the solution is heated at a temperature of no less than about 40° C., and more preferably at about reflux temperature. The amount of time the solution is heated may vary depending on, for example, the temperature, solvent volume, or amount of reagents. After deprotection, the solution may be filtered to remove any remaining solids.

The second portion of water is added to form an aqueous phase and an organic phase, which are then separated. Any amount of water sufficient to form an aqueous phase may be added. This volume of water may be added all in one step, or it may be added in separate aliquots. Preferably, the first portion of water is present in an amount of about 0.5 ml/g to about 5 ml/g of the trityl candesartan cilexetil, more preferably about 1 ml/g. Preferably, the second portion of water is present in an amount of about 0.5 ml/g to about 5 ml/g of the trityl candesartan cilexetil, more preferably about 4 ml/g. Preferably, the total amount of water is about 4 ml/g to about 6 ml/g of the trityl candesartan cilexetil, more preferably about 5 ml/g.

In one embodiment, the aqueous phase may be extracted with multiple portions of water immiscible solvent. After extraction, candesartan cilexetil is recovered from the organic phase.

Recovery of the candesartan cilexetil from the organic phase may be by filtration, evaporation, or any other methods commonly used. Additionally, the candesartan cilexetil may be purified by any method known in the art, such as column chromatography or crystallization.

Recovery of candesartan cilexetil may be from the organic phase of the two-phase system. Furthermore, candesartan cilexetil may be recovered by separating the two-phase system in a continuous counter current, co-current, or cross current extraction to obtain the candesartan cilexetil.

The candesartan cilexetil may be isolated at room temperature. Preferably, the phase containing the candesartan cilexetil is cooled. More preferably the phase containing the candesartan cilexetil is cooled at a temperature of about −10° C. to about 10° C., and most preferably at about 0° C.

Having described the invention with reference to certain preferred embodiments, other embodiments will become apparent to one skilled in the art from consideration of the specification. The invention is further defined by reference to the following example describing in detail the process of preparing candesartan cilexetil. It will be apparent to those skilled in the art that many modifications, both to materials and methods, may be practiced without departing from the scope of the invention.

### EXAMPLES

### Example 1

A solution of trityl candesartan cilexetil (70 g, 82 mmol), toluene (210 ml), methanol (420 ml), and water (3.5 ml) was refluxed for about 4.5 hours. The clear solution was cooled, filtered, and the filtrate was returned to the reactor. Water (350 ml, 5 ml/g trityl candesartan cilexetil) was added, and the solution was stirred for a few minutes, giving two liquid phases after the mixing was stopped. The bottom phase (toluene, 225.5 g) was collected to a vessel, while the upper phase was left in the reactor. Toluene (70 ml, 1 ml/g trityl candesartan cilexetil) was added to the upper phase, and the solution was stirred for a few minutes, giving two liquid phases after the mixing was stopped

The reactor was emptied from the methanol-water phase (now in the bottom), the toluene phase (80 g) was added to the first toluene phase, and the combined phase was returned to the reactor. The reactor was cooled to 0° C., stirred for 16 hours, and filtered. The solids were washed with toluene (1 ml/g trityl candesartan cilexetil) to give 43.5 g on dry basis. Yield: 86% by weight.

### Example 2

A solution of trityl candesartan cilexetil (TCS, 70 g, 82 mmol), toluene (210 mL), methanol (420 mL) and water (3.5 mL) was refluxed for about 4 h. The clear solution was cooled and filtered. The filtrate was returned to the reactor and 2 volumes of water were added (140 mL). The solution was stirred for a few minutes, giving two liquid phases after the mixing was stopped. The bottom phase (toluene, 220.5 g) was collected to a vessel, while the upper phase was left in the reactor. 1 volume of toluene (70 mL) and 1 volume of water (70 mL) were added to the upper phase. The solution was stirred for a few minutes, giving two liquid phases after the mixing was stopped.

The reactor was emptied from the methanol-water phase (now in the bottom) and the toluene phases (52 g) were added to the first toluene phase, and the combined phase was returned to the reactor. The reactor was cooled to 0° C., stirred for 17 hours, and filtered. The solids were washed with 1 volume of toluene to give 47.5 g on dry basis. Yield: 94% by weight.

### Example 3

A solution of trityl candesartan cilexetil (TCS, 50 g, 59 mmol), toluene (150 mL), methanol (300 mL) and water (2.5 mL) was refluxed for about 4.5 h. The clear solution was cooled and filtered. The filtrate was returned to the reactor and 2 volumes of water were added (100 mL). The solution was stirred for a few minutes, giving two liquid phases after the mixing was stopped. The bottom phase (toluene, 147 g) was collected to a vessel while the upper phase was left in the reactor. 3 volumes of toluene (150 mL) were added to the upper phase. The solution was stirred for a few minutes, giving two liquid phases after the mixing was stopped.

The reactor was emptied from the methanol-water phase (now in the bottom) and the toluene phases (140 g) were added to the first toluene phase, and the combined phase was returned to the reactor. The reactor was cooled to 0° C., stirred for 24 hours, and filtered. The solids were washed with 1 volume of toluene to give 27.9 g on dry basis. Yield: 83% by weight.

What is claimed is:

1. A process for preparing candesartan cilexetil comprising:
a. heating a solution of trityl candesartan cilexetil in a water immiscible solvent in the presence of at least one C₁-C₄ alcohol and a first portion of water;
b. combining the solution with a second portion of water to obtain a two-phase system; and
c. recovering candesartan cilexetil.

2. The process according to claim 1, wherein the C₁-C₄ alcohol is methanol, ethanol, propanol, isopropanol, butanol, or 2-butanol.

3. The process according to claim 1, wherein the C₁-C₄ alcohol is methanol.

4. The process according to claim 1, wherein the C₁-C₄ alcohol is present in an amount of about 4 ml/g to about 12 ml/g of the trityl candesartan cilexetil.

5. The process according to claim 1, wherein the C₁-C₄ alcohol is present in an amount of about 6 ml/g of the trityl candesartan cilexetil.

6. The process according to claim 1, wherein the water immiscible solvent is at least one of halo-hydrocarbons, C₆₋₁₀ aromatic hydrocarbons, linear or cyclic C₂₋₅ alkyl ethers, esters, C₃₋₅ ketones, C₁₋₅ amides, or carbonates.

7. The process according to claim 1, wherein the water immiscible solvent is methylene chloride, ethyl acetate, or toluene.

8. The process according to claim 1, wherein the water immiscible solvent is toluene.

9. The process according to claim 1, wherein the water immiscible solvent is present in an amount of about 1 mL/g to about 6 mL/g of the trityl candesartan cilexetil.

10. The process according to claim 1, wherein the water immiscible solvent is in an amount of about 3 mL/g of the trityl candesartan cilexetil.

11. The process according to claim 1, wherein the first portion of water is present in an amount of at least about 0.5 mole per mole of trityl candesartan cilexetil.

12. The process according to claim 1, wherein the first portion of water is present in an amount of about 2 mole equivalents of the trityl candesartan cilexetil.

13. The process according to claim 1, wherein the second portion of water is present in an amount of about 0.5 mL/g to about 5 mL/g of the trityl candesartan cilexetil.

14. The process according to claim 1, wherein the second portion of water is added in an amount of about 4 mL/g of the trityl candesartan cilexetil.
